(19) United States
(12) Patent Application Publication
(10) Pub. No.: US 2006/0286159 A1
(43) Pub. Date: Dec. 21, 2006

(54) Treatment of persistent active tendinopathy using transdermal glyceryl trinitrate providing durability of effect

(76) Inventors: George Anthony Calvert Murrell, Sidney (AU); Robert Ang, New York, NY (US); Sven Jacobson, New York, NY (US); David Geliebter, Franklin Lakes, NJ (US)

Correspondence Address: THORPE NORTH & WESTERN, LLP., 8180 SOUTH 700 EAST, SUITE 200, SANDY, UT, 84070, US

(21) Appl. No.: 11/446,571

(22) Filed: Jun. 1, 2006

Related U.S. Application Data
(63) Continuation-in-part of application No. 10/967,707, filed on Oct. 15, 2004.
(60) Provisional application No. 60/512,070, filed on Oct. 17, 2003.

(51) Int. Cl. A61K 9/70; A61K 31/21

(52) U.S. Cl. 424/449; 514/509

(57) ABSTRACT

The present invention provides methods for treating tendinopathy providing a durability of effect by administering nitroglycerin. Such methods include methods for relieving pain associated with such tendinopathies. The use of a transdermal patch configured to deliver glyceryl trinitrate at a rate of 5 mcg/hr to about 85 mcg/hr

The present application is a continuation-in-part of U.S. patent application Ser. No. 10/967,707, filed Oct. 15, 2004, which claims the benefit of U.S. Provisional Patent Application Ser. No. 60/512,070, filed Oct. 17, 2003, each of which is incorporated herein by reference in its entirety.

### FIELD OF THE INVENTION

The present invention relates to the treatment of persistent active tendinopathies, including overuse tendinopathy and chronic overuse tendinopathy, using transdermally administered glyceryl trinitrate.

### BACKGROUND OF THE INVENTION

Various forms of tendinopathy are common causes of discomfort and pain for many people. There are a number of areas of the body where tendinopathy can occur but some forms are particularly common. Perhaps the most common is Extensor tendinosis. Extensor tendinosis (“tennis elbow” or lateral epicondylitis) is a degenerative overuse tendinopathy of the wrist extensors at their attachment to the lateral humeral epicondyle. No treatment has been universally successful in managing this condition. Although it is frequently referred to as “tennis elbow,” extensor tendinosis is not restricted to tennis players. People at risk of this condition involve those who participate in repetitive upper limb activities involving flexing and extension of the wrist. This includes workers with many occupations such as, for example, carpenters, painters, process workers, and participants in racquet sports, golf, and throwing sports.

Tennis elbow is typically caused by overuse of the tendons which extend the wrist. This causes damage to the tendon at its site of attachment into the elbow. The cellular events that lead to tendon damage are undetermined. Ninety percent of people with tennis elbow develop pain on and around the bony prominence (epicondyle) on the outside (lateral side) of the elbow. The pain is usually exacerbated by activities such as lifting objects, unscrewing jars, playing golf or tennis, and repetitive movements such as painting or hammering nails. In chronic cases, pain may be present with writing and shaking hands and many people describe “aching” of the elbow while at rest.

In addition to tennis elbow, other common degenerative tendinopathies associated with overuse include non-insertional Achilles tendinopathy and rotator cuff tendinopathy. Non-insertional Achilles tendinopathy is especially common among runners, and rotator cuff tendon injury, such as supraspinatus tendinopathy, is prevalent in overhead workers (e.g., painters) and throwing athletes.

There are a variety of non-operative treatments for tendinopathy, many with unproven therapeutic efficacy, and none that are universally effective in the management of chronic tendinopathies. The non-operative management of tendinopathy involves rehabilitation consisting of relative rest, stretching, and a graduated strengthening exercise program focusing on eccentric tendon loading. In some cases, braces can be useful in reducing the force transmitted to the tendon at the joint. Splints to block extension also can be useful by enabling the tendons to rest. Oral anti-inflammatory medications can be useful in some cases and corticosteroid injections can be useful in chronic cases by reducing the pain, thereby enabling a person to perform the rehabilitation exercises.

Relative rest may be one aspect of tendon rehabilitation as suggested by recent research on the role of stress activated protein kinases in apoptosis in degenerative tendinopathies. Tendon unloading with heel-raises has been advocated for treating Achilles tendinopathy. Corticosteroid injections remain controversial, and there little evidence that they produce more than a short term therapeutic effect.

Nitric oxide (NO) is endogenously produced by three isoforms of the enzyme nitric oxide synthase, inducible nitric oxide synthase (iNOS), an isoform originally found in endothelial cells (eNOS), and an isoform originally found in brain tissue and neuronal cells (bNOS). NO is produced in large amounts by inflammatory cells such as macrophages, neutrophils, lymphocytes and peripheral-blood monocytes during immunological reactions and septic shock. There is also an inducible form of nitric oxide synthase in cartilage.

### SUMMARY OF THE INVENTION

The present invention describes the unexpected durability of effect benefit of glyceryl trinitrate. In one embodiment, a method of treating a subject experiencing tendinopathy involves administering to a skin site proximate an affected tendon of the subject a glyceryl trinitrate-containing transdermal patch. The administration of the glyceryl trinitrate can be for an administration period of from 1 to 52 weeks, during which period the patch can be replaced. The transdermal patch can be configured to deliver glyceryl trinitrate at a rate of from 5 mcg/hr to about 85 mcg/hr. The clinical and therapeutic effects associated with the administration of the patch continue for a durability of effect period of at least as long as the administration period. In one embodiment, the method of treating the subject includes a method of relieving pain caused by tendinopathy.

In another embodiment, the invention provides a transdermal patch for the delivery of a nitroglycerin. The transdermal patch comprises a backing layer and a glyceryl trinitrate-containing composition supported at least in part by the backing layer. The transdermal patch is formulated to deliver nitroglycerin at from about 5 μg/hour to about 85 μg/hour and configured to be administered to a subject during an administration period of from 4 to 52 weeks. Upon termination of the administration of the transdermal patch, the clinical and therapeutic effects against persistent active tendinopathy continue for a durability of effect period of at least as long as the administration period.

### BRIEF DESCRIPTION OF THE DRAWINGS

FIG. 1

1(a) depicts the effects of glyceryl trinitrate 1.25 mg/day (˜52 mcg/hr) via transdermal patch plus rehabilitation (GTN, n=41) versus rehabilitation alone (placebo, n=43) on Achilles tendon pain with activity. Statistically significant differences between groups are shown with an asterisk (*p<0.05).

1(b) depicts effects of glyceryl trinitrate 1.25 mg/day (˜52 mcg/hr) via transdermal patch plus rehabilitation (GTN, n=47) versus rehabilitation alone (placebo, n=48) on lateral elbow pain with activity in extensor tendinopathy. Statistically significant differences between groups are shown with an asterisk (*p<0.05).

1(c) shows effects of glyceryl trinitrate 1.25 mg/day (˜52 mcg/hr) via transdermal patch plus rehabilitation (GTN, n=28) versus rehabilitation alone (placebo, n=29) on shoulder pain with activity in supraspinatus tendinopathy. Statistically significant differences between groups are shown with an asterisk (*p<0.05).

FIG. 2

2(a) shows effects of glyceryl trinitrate (GTN, n=41) 1.25 mg/day (˜52 mcg/hr) via transdermal patch, plus rehabilitation versus rehabilitation alone (placebo, n=43), on ORI-ASTS measured ankle plantarflexor mean total work (Achilles tendinopathy). These results are expressed as increases from baseline as there was a significant difference in mean total work at week 0. Statistically significant differences between groups are shown with an asterisk (*p<0.05).

2(b) shows effects of glyceryl trinitrate (GTN, n=47) 1.25 mg/day (˜52 mcg/hr) via transdermal patch, plus rehabilitation versus rehabilitation alone (placebo, n=48), on ORI-TETS measured mean total work (tennis elbow). Statistically significant differences between groups are shown with an asterisk (*p<0.05).

2(c) shows effects of glyceryl trinitrate (GTN, n=28) 1.25 mg/day (˜52 mcg/hr) via transdermal patch plus rehabilitation, versus rehabilitation alone (placebo, n=29), on dynamometer measured supraspinatus force (supraspinatus tendinopathy). Statistically significant differences between groups are shown with an asterisk (*p<0.05, **p<0.01).

FIG. 3

3(a) depicts effects of glyceryl trinitrate (GTN, n=41) 1.25 mg/day (˜52 mcg/hr) via transdermal patch plus rehabilitation versus rehabilitation alone (placebo, n=43) on pain scores after the 10 hop test (Achilles tendonitis). Statistically significant differences between groups are shown with an asterisk (*p<0.05, **p<0.01).

3(b) shows effects of glyceryl trinitrate (GTN, n=28) 1.25 mg/day (˜52 mcg/hr) via transdermal patch versus rehabilitation alone (placebo, n=29) on shoulder impingement in internal rotation. Statistically significant differences are shown with an asterisk (*p<0.05).

3(c) demonstrates effects of glyceryl trinitrate (GTN, n=28) 1.25 mg/day (˜52 mcg/hr) via transdermal patch versus rehabilitation alone (placebo, n=29) on passive shoulder abduction range of motion. Statistically significant differences are shown with an asterisk (*p<0.05).

FIG. 4

4(a) shows the percentage differences in mean grouped outcome measures between the glyceryl trinitrate group (GTN 1.25 mg/day patch (˜52 mcg/hr), n=41) and the placebo patch group (n=43); a between group comparison of means for grouped outcome measures in the Achilles tendinopathy clinical trial.

4(b) shows the percentage differences in mean grouped outcome measures between the glyceryl trinitrate group (GTN 1.25 mg/day patch (˜52 mcg/hr), n=47) and the placebo patch group (n=48); a between group comparison of means for grouped outcome measures; a between group comparison of means for grouped outcome measures in the extensor tendinopathy clinical trial.

4(c) shows the percentage differences in mean grouped outcome measures between the glyceryl trinitrate group (GTN 1.25 mg/day patch (˜52 mcg/hr), n=28) and the placebo patch group (n=29); a between group comparison of means for grouped outcome measures; a between group comparison of means for grouped outcome measures in the supraspinatus tendinopathy clinical trial.

### DETAILED DESCRIPTION OF THE PREFERRED EMBODIMENT(S)

The present invention provides a method for treating tendinopathy which comprises administering an effective amount of glyceryl trinitrate for a period of from 1 to 52 weeks at a skin site proximate an affected tendon. After the administration is complete, the clinical and therapeutic results of the administration continue for a durability of effect period of at least as long as the administration period. During administration the patch is placed directly on a skin surface that is proximate the affected tendon, and can be replaced periodically during the administration period to improve force and functional outcome measures at the affected tendon, and/or to relieve pain. In one embodiment, the patch is replaced daily (every 24 hours). In another embodiment, a new or replacement patch is placed on a different or new skin site which is also proximate the affected tendon. The present invention exemplifies treating three different chronic overuse tendinopathies using a transdermal patch delivering a significantly less glyceryl trinitrate than the patch that is marketed and indicated for the treatment of angina.

The term “glyceryl trinitrate” refers to 1,2,3-trinitroglycerin, 1,2,3-propanetriol trinitrate, or nitroglycerin, CAS No. 55-63-0 (GTN).

As used herein, the term “affected tendon” refers to a tendon that is characterized by pain or tenderness, which can be in the absence of inflammation, and is the subject of a diagnosis of tendinopathy according to those skilled in the art, such as described herein. The diagnosis can usually be made by clinical methods e.g., taking a history regarding the problem and examining the patient, and may be aided by soft tissue imaging studies for example, by ultrasound or MRI. The tendinopathy can be acute or chronic tendinopathy, where “acute” generally includes a duration of symptoms from days to weeks, and “chronic” generally includes a duration of symptoms from months to years.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value.

A “subject,” “patient,” or “mammal” is an animal that has developed, or is developing acute or chronic tendinopathy, including but not limited to extensor tendinopathy (tennis elbow), Achilles tendinopathy, supraspinatus tendinopathy (rotator cuff), patellar tendinopathy, quadriceps tendinopathy, hip adductor tendinopathy, common flexor tendinopathy of the elbow (golfer's elbow), and/or tendinopathy of the thumb. The animal is typically a mammal, and is often a human.

The terms “treat” or “treatment” means to therapeutically intervene in the development of a disease or disorder in a subject showing a symptom of this disease, e.g., tendinopathy. In the context of the present invention, these symptoms can include but are not limited to, pain or tenderness in the affected tendon, limited range of motion or ability to exert a force on the affected tendon without pain, aching of the affected tendon at rest, with activities, and/or at night.

The terms “improve function,” “improved function,” or “improving function” as used herein include significant increases in force outcome measures at the affected tendon, as determined by routine methods in the art, including but not limited to the Orthopaedic Research Institute-Ankle Strength Testing System (ORI-ASTS), and dynamometer and Tennis Elbow Testing System (ORI-TETS). These tests measure increases in mean total work, and increases in dynamometer resisted force measurements for the affected tendons. These terms can also include significant increases in functional outcome measures. Function can be determined by, but is not limited to, the 10 hop test for non-insertional Achilles tendinopathy (similar to tests in the newly validated VISA-A Achilles tendon scale), the ORI-TETS mean peak force and mean total work for extensor tendinopathy, and shoulder passive range of motion in abduction and in internal rotation, as well as shoulder impingement in internal rotation and strength as determined by a hand held dynamometer for supraspinatus tendinopathy. Hopping involves Achilles tendon loading through push-off and landing as used in running and jumping; wrist extensor tendon peak force and total work are measured with a modified chair pick-up test (ORI-TETS). Other similar tests for various body parts that are known in the art are also included for the purpose of testing improved function. Increases in functional outcome also refer to a subject treated according to the method of the present invention becoming asymptomatic with activities of daily living.

The terms “relieve pain” or “relieving pain” include improved patient rated pain scores as determined, for example, using the Mann-Whitney rank sum tests. In the context of the present invention, this also refers to subjective determinations such as decreased tenderness at the affected tendon or joint, decreased night pain at the affected tendon or joint, and/or decreased pain with activity at the affected tendon or joint.

The phrase “pharmaceutically acceptable” refers to molecular entities and compositions that are generally regarded as safe, e.g., that are physiologically tolerable and do not typically produce an allergic or similar untoward reaction, such as gastric upset, dizziness, and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to diluents, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. Generally any known pharmaceutical carrier can be used in the present invention so long as it does not negatively impact the effectiveness of the active agent being administered.

The terms “continuous” or “continuously” in the context of drug administration refers to a constant, pre-determined amount of drug that is administered over a specified dosing period. A dosing period is the time during which one of the dosage forms in the series is administered to the patient. Accordingly, the dosing regimen will consist of a separate dosing period for administration of each dosage form in the series. Thus, for example, the first dosage form in the series may be worn by the patient for 24 consecutive hours. As one example, as used herein, continuous administration refers to delivery of 1.25 mg of glyceryl trinitrate to a subject over 24 hours via a transdermal patch, for successive 24 hour periods for 12-24 weeks. In this context, continuous administration of the preceding transdermal patch requires replacing the patch every 24 hours. Other replacement regimens can also be carried out, depending on the dosages and length of time that a particular patch is designed to last.

The terms “relative release rate,” “flux rate,” or “delivery rate” are determined from the amount of drug released per unit time from e.g., a transdermal delivery system through the skin and into the bloodstream of a subject. Mean relative release rate may be expressed, e.g., as mcg (μg) drug/hr or, for comparing delivery systems covering skin areas of different size, as mcg (μg) drug/cm²/hr. For example, a transdermal delivery system that releases 1.25 mg of glyceryl trinitrate over a time period of 24 hours is considered to have a relative release rate of about 52.1 mcg/hr. Even in this specific embodiment, the release profile per cm² can vary, depending on the design of the particular patch. For purposes of the invention, it is understood that relative release rates may change between any particular time points within a particular dosing interval, and the term therefore only reflects the overall or average release rate during the particular dosing interval.

As used herein, the term “administration period” refers to the period of time in which nitroglycerin is actively administered to a subject. In certain non-limiting exemplary embodiments, the administration period can be from about 1 to about 52 weeks. In one embodiment, the administration period can be from about 4 to about 24 weeks. In another embodiment, the administration period can be from about 6 to about 12 weeks. In another embodiment, the administration period can be about 8 weeks.

As used herein, the term “durability of effect” refers to the continuation of at least one of the clinical and/or therapeutic effects of the nitroglycerin after discontinuing the administration thereof. Such continuation of the clinical and therapeutic effects can last for a period of time at least as long as the administration period of nitroglycerin. The term “durability of effect period” refers to the period beginning immediately following the administration period. This period occurring after the administration period relates is characterized by no glyceryl trinitrate being administered, but where the therapeutic and clinical effects of the glyceryl trinitrate administration still continue. Depending on the dosage of the glyceryl trinitrate as well as the length of the administration, the durability of effect period lasts at least as long as the administration period, but can last up to about 5 or more times the length of the administration period. In one embodiment, the durability of effect period is at least three or more months. In another embodiment, the durability of effect period is at least a year.

The term “clinical and therapeutic effects” refers to certain physical outcome measures including but not limited to reduced pain upon activity, reduced night pain, reduced pain at rest, reduced tenderness, improved strength, improved function, increased range of motion, and combinations thereof. The manifestations of the clinical and therapeutic effects can vary depending on the subject and the precise nature of the tendinopathy.

The phrase “persistent active tendinopathy” refers to tendinopathy in any of the herein described forms which, despite treatment, continues to have at least some negative or undesirable effects on a subject. The negative or undesirable effects can include pain upon activity, night pain, pain at rest, tenderness, reduced strength, diminished functionality, decreased range of motion, or combinations thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

The present invention provides methods for treating tendinopathy, in particular persistent active tendinopathy, which provide for durability of effect periods which are at least as long as the administration period of glyceryl trinitrate. The clinical and therapeutic effects which are extended from the administration period into the durability of effect period can include, but are not limited to, reduction of pain, reduction of night pain, reduction of pain at rest, reduction of pain upon activity, reduction of tenderness, improved function, or combinations thereof.

For the purposes of the present invention, the administration period can be from about 1 to about 52 weeks in duration. In one embodiment, the administration period is from about 4 to about 24 weeks. In another embodiment, the administration period can be from about 6 to about 12 weeks. In another embodiment, the administration period can be 8 weeks. As discussed above, with each administration period there is a corresponding durability of effect period which lasts at least as long as the administration period. In some cases, the durability of effect period can last 5 or more times as long as the administration period. For example, in one embodiment, when glyceryl trinitrate is administered continuously for an administration period of 24 weeks, the durability of effect period can be 24 weeks to as long as 120 weeks or more. When the durability of effect period is 4-24 weeks the durability of effect period can be from at least 4 weeks to 120 weeks or more, for example.

Transdermal dosage forms are convenient dosage forms for delivering many different active therapeutically effective agents, including but not limited to glyceryl trinitrate. Transdermal dosage forms are particularly useful for timed release or sustained release of active agents.

Transdermal dosage forms may be classified into transdermal dosage articles and transdermal dosage compositions. The most common transdermal dosage article is a diffusion driven transdermal system (transdermal patch) using either a fluid reservoir or a drug in adhesive matrix system. Transdermal dosage compositions include, but are not limited to, topical gels, lotions, ointments, transmucosal systems and devices, and iontophoretic (electrical diffusion) delivery systems. Preferably, for the method of the present invention, the transdermal dosage form is a transdermal patch. The transdermal dosage form is used in the dosage regimen of the present invention for timed release or sustained release of glyceryl trinitrate.

Transdermal patches used in accordance with the invention preferably include a backing layer made of a pharmaceutically acceptable material which is impermeable to the glyceryl trinitrate. The backing layer preferably serves as a protective cover for the glyceryl trinitrate, and may also provide a support function. Examples of materials suitable for making the backing layer are films of high and low density polyethylene, polypropylene, polyvinylchloride, polyurethane, polyesters such as poly(ethylene phthalate), metal foils, metal foil laminates of such suitable polymer films, textile fabrics (if the components of the reservoir cannot penetrate the fabric due to their physical or other properties), and the like. Usually, the materials used for the backing layer are laminates of such polymer films with or without a metal foil such as aluminum foil. The backing layer can be any appropriate thickness to provide the desired protective and support functions. A suitable thickness will be from about 10 microns to about 200 microns. Desirable materials and thickness will be apparent to the skilled artisan.

In certain preferred embodiments, the transdermal dosage forms used in accordance with the invention contain a pharmacologically or biologically acceptable polymer matrix layer. Generally, the polymers used to form the polymer matrix are those capable of forming thin walls or coatings through which pharmaceuticals can pass at a controlled rate. General categories of typical polymer matrices include silicon-based adhesives, acrylic or acrylate based adhesives, and/or PIB-based adhesives. A non-limiting list of exemplary materials for inclusion in the polymer matrix includes polyethylene, polypropylene, ethylene/propylene copolymers, ethylene/ethylacrylate copolymers, ethylenevinyl acetate copolymers, silicones, rubber, rubber-like synthetic homo-, co- or block polymers, polyacrylic esters and the copolymers thereof, polyurethanes, polyisobutylene, chlorinated polyethylene, polyvinylchloride, vinyl chloride-vinyl acetate copolymer, polymethacrylate polymer (hydrogel), polyvinylidene chloride, poly(ethylene terephthalate), ethylene-vinyl alcohol copolymer, ethylene-vinyloxyethanol copolymer, silicones including silicone copolymers such as polysiloxane-polymethacrylate copolymers, cellulose polymers (e.g., ethyl cellulose, and cellulose esters), polycarbonates, polytetrafluoroethylene, or the like, or mixtures thereof. Other exemplary materials for inclusion in the polymer matrix layer are silicone elastomers of the general polydimethylsiloxane structures, (e.g., silicone polymers). Preferred silicone polymers cross-link and are pharmaceutically or biologically acceptable.

Other preferred materials for inclusion in the polymer matrix layer include: silicone polymers that are cross-linkable copolymers having dimethyl and/or dimethylvinyl siloxane units that can be crosslinked using a suitable peroxide catalyst. Also preferred are those polymers consisting of block copolymers based on styrene and 1,3-dienes (particularly linear styrene-isoprene-block copolymers of styrene-butadiene-block copolymers), polyisobutylenes, polymers based on acrylate and/or methacrylate.

The polymer matrix layer may optionally include a pharmaceutically acceptable crosslinking agent. A non-limiting example of suitable crosslinking agent is tetrapropoxy silane. Transdermal delivery systems used in accordance with the methods of the present invention can include an adhesive layer to affix the dosage form to the skin of the patient for the desired period of administration. If the adhesive layer of the dosage form fails to provide adhesion for the desired period of time, it is possible to maintain contact between the dosage form and the skin by, for instance, affixing the dosage form to the skin of the patient with an adhesive tape, e.g., surgical tape.

The adhesive layer preferably includes using any adhesive known in the art that is pharmaceutically compatible with the dosage form. The dosage form and matrix layer can also be hypoallergenic, and can include polymers such as polyacrylic adhesive polymers, acrylate copolymers (e.g., polyacrylate) and polyisobutylene adhesive polymers. In other embodiments of the invention, the adhesive is a hypoallergenic and pressure-sensitive contact adhesive.

The transdermal dosage forms that can be used in accordance with the present invention may optionally include a permeation enhancing agent. Permeation enhancing agents are compounds that promote penetration and/or absorption of the glyceryl trinitrate through the skin or mucosa and into the blood stream of the patient. A non-limiting list of permeation enhancing agents includes polyethylene glycols, surfactants, and the like. Alternatively, permeation of the active agent such as glyceryl trinitrate may be enhanced by occlusion of the dosage form after application to the desired site on the patient with, e.g. an occlusive bandage. Permeation may also be enhanced by removing hair from the application site by, e.g. clipping, shaving or use of a depilatory agent. Another permeation enhancer is heat. It is thought that permeation can be enhanced by, among other things, the use of a radiating heat form, such as an infrared lamp, at the application site during at least a portion of the time the transdermal dosage form is applied on the skin or mucosa. Other means of enhancing permeation of the active agent, such as the use of iontophoretic means, are also contemplated to be within the scope of the present invention. This being stated, in embodiments where a low dose of nitroglycerin is delivered, permeation enhancement may not be desirable for use; as such resulting effects may be unnecessary. However, when warranted or desired, strategies of permeation enhancement can be included in accordance with embodiments of the present invention.

The active agent, namely glyceryl trinitrate, may be included in the device in a drug reservoir, drug matrix, or drug/adhesive layer. This area of the patch and the amount of active agent per unit area determine the limit dose, as one of ordinary skill in the art can readily determine.

Certain preferred transdermal delivery systems also include a softening agent in the reservoir or matrix. Suitable softening agents include higher alcohols such as dodecanol, undecanol, octanol, esters of carboxylic acids, wherein the alcohol component may also be a polyethoxylated alcohol, diesters of dicarboxylic acids, such as di-n-butyladiapate, and triglycerides, particularly medium-chain triglycerides of caprylic/caproic acids or coconut oil. Further examples of suitable softeners are, for example, multivalent alcohols such as glycerol and 1,2-propanediol, as well as softeners such as levulinic acid and caprylic acid, which can also be esterified by polyethylene glycols.

Transdermal dosage systems are described generally in U.S. Pat. No. 6,231,885 to Carrara; U.S. Pat. No. 5,948,233 to Burton; U.S. Pat. No. 5,324,521 to Gertner; and U.S. Pat. No. 5,310,559 to Shah et al, the teachings of which are incorporated herein by reference to the extent compatible with the teachings of the present invention.

Commercially available transdermal glyceryl trinitrate dosage forms include Deponit™ (Schwarz), Minitran™ (3M), Nitro-Dur™ (Schering-Plough), Percutol™ (Dominion), Transiderm-Nitro™ (Novartis), and Trintek™ (Goldschield). For example, the Nitro-Dur™ patch is a transdermal infusion system that provides continuous controlled-release through intact skin. The Nitro-Dur patches come with varying delivery rates ranging from 0.1 mg/hr to 0.8 mg/hr, and such patches can contain from 20 mg of nitroglycerin to 160 mg of nitroglycerin in an acrylic-based polymer adhesive with resinous cross-linking agent to provide continuous administration. The rate of release is generally linear, depending on the area of the patch, with each cm² of applied patch delivering approximately 0.02 mg/hour. Thus, the patch containing 40 mg patch delivers approximately 0.1 mg/hr over a patch area of 10 cm². Each transdermal patch unit is sealed in a paper polyethylene-foil pouch.

Patches containing glyceryl trinitrate are further described in U.S. Pat. No. 5,762,952 to Barnhart; U.S. Pat. No. 5,613,958 to Kochinke et al.; U.S. Pat. No. 5,252,165 to Govil; and U.S. Pat. No. 4,615,699 to Gale et al.; the teaching of which are incorporated herein by reference to the extent the teachings are compatible with the present invention.

In addition to transdermal patches, the present invention contemplates the use of any topical dosage form known in the art. Such dosage forms include topical solutions, suspensions, ointments, pastes, creams, lotions, gels, and the like. Preparations of such dosage forms are well known in the art and can be formulated using numerous known excipients.

Such pharmaceutically acceptable excipients include polymers, oils, liquid carriers, surfactants, buffers, preservatives, stabilizers, antioxidants, moisturizers, emollients, colorants, odorants, and mixtures thereof.

Examples of pharmaceutically acceptable polymers suitable for such topical formulations include, but are not limited to, acrylic polymers, cellulose derivatives, such as carboxymethylcellulose sodium, methylcellulose or hydroxypropylcellulose; natural polymers, such as alginates, tragacanth, pectin, xanthan, cytosan, and mixtures thereof.

Examples of suitable pharmaceutically acceptable oils which are so useful include but are not limited to, mineral oils, silicone oils, fatty acids, alcohols, glycols, and mixtures thereof.

Examples of suitable pharmaceutically acceptable liquid carriers include, but are not limited to, water, alcohols or glycols such as ethanol, isopropanol, propylene glycol, hexylene glycol, glycerol and polyethylene glycol, and mixtures thereof, or other mixtures in which the pseudopolymorph is dissolved or dispersed, optionally with the addition of non-toxic anionic, cationic or non-ionic surfactants, inorganic or organic buffers, and mixtures thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, parabens (such as methyl paraben, ethyl paraben, propyl paraben, etc.), and mixtures thereof.

Suitable examples of pharmaceutically acceptable stabilizers and antioxidants include, but are not limited to, ethylenediaminetetraacetic acid (EDTA), thiourea, tocopherol, butyl hydroxyanisole, and mixtures thereof.

Suitable examples of pharmaceutically acceptable moisturizers include, but are not limited to, glycerine, sorbitol, urea, polyethylene glycol, and mixtures thereof.

Suitable examples of pharmaceutically acceptable emollients include, but are not limited to, mineral oils, isopropyl myristate, isopropyl palmitate, and mixtures thereof.

The use of dyes and odorants in topical formulations of the present invention depends on many factors including organoleptic acceptability to the population that will be using the pharmaceutical formulations.

Whether administered as part of a transdermal patch or other topical formulation, the dosage forms used in the method of the present invention may be administered alone or in combination with other active agents, e.g., such as an analgesic or anti-inflammatory, including, for example, a non-steroidal anti-inflammatory drug (NSAID) such as acetaminophen, ibuprofen, or acetylsalicylic acid. For combination treatment with more than one active agent, where the active agents are in separate dosage formulations, the active agents can be administered concurrently, or they each can be administered at separately staggered times. The dosage amount may be adjusted when combined with other active agents as described above to achieve desired effects. Alternatively, unit dosage forms of these various active agents may be independently optimized and combined to achieve an improved result wherein the pathology is reduced more than it would be if either active agent were used alone. In some embodiments, synergy can be present. In most cases, depending on the secondary active agent administered with the glyceryl trinitrate, the durability of the effect of the secondary active agent is not as great as the effect of the glyceryl trinitrate, though variability occurs from case-by-case.

The dosage of glyceryl trinitrate according to the present invention can be determined on an individual, case-by-case basis by one of ordinary skill in the art, but in one embodiment, the transdermal patch will generally not be formulated to exceed a delivery rate of about 85 mcg/hr. In one embodiment, the transdermal patch will deliver from about 5 mcg/hr to about 85 mcg/hr of glyceryl trinitrate. In another embodiment, the transdermal patch will deliver from about 15 mcg/hr to about 75 mcg/hr of glyceryl trinitrate. In still another embodiment, the transdermal patch will deliver from about 30 mcg/hr to about 65 mcg/hr of glyceryl trinitrate.

During the administration period of the glyceryl trinitrate, the transdermal patch can be replaced with a similar new patch. For the purposes of this invention, the replacement of a transdermal patch with a new similar glyceryl trinitrate patch does not disrupt the continuity of the administration period. For example, if a new patch is administered to the same or similar region of skin proximate to an affected tendon every three days for a period of 8 weeks, for the purposes of the present invention, the administration period would be considered to be continuous and have a length of eight weeks. An eight week administration period of glyceryl trinitrate would provide for a durability of effect period lasting at least 8 weeks, though a durability of effect of 40 weeks or longer may also be achieved in some subjects.

### EXAMPLES

The following examples illustrate exemplary embodiments of the invention. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what is presently deemed to be practical embodiments of the invention.

### Example 1

The following demonstrates that glyceryl trinitrate at 1.25 mg/24 hour (about 52.1 mcg/hr), has clinically demonstrated efficacy in modulating pain, force measures, functional measures, and patient outcomes at three and six months in three common chronic overuse tendinopathies.

Three clinical trials were approved by an institutional Ethics Committee. Patients with clinical diagnoses of the specified tendinopathies were recruited through newspaper advertisements and private consulting rooms. All subjects were over 18 years of age, and gave written informed consent.

In the non-insertional Achilles tendinopathy trial, there were 65 patients (84 Achilles tendons) with 40 men and 25 women enrolled in the study, having a median age of 49 years (range 24 to 77 years), and a median duration of symptoms prior to the study of 16 months (range 4 to 147 months). In the extensor tendinopathy trial, there were 86 patients (95 elbows), with 42 males and 44 females, having a median age of 46 years (range 30 to 74 years), and a median duration of symptoms of 17 months (range 3 to 232 months). In the supraspinatus tendinopathy trial, there were 53 patients (57 shoulders), with 24 males and 29 females, having a median age of 52 years (range 25 to 79 years), and a median symptom duration of 14 months (range 4 to 96). In all trials, there were no significant differences between groups with respect to average age, sex, affected side, symptom severity, or symptom duration.

Diagnostic criteria for patient inclusion in the respective trials were as follows. The diagnosis of chronic non-insertional Achilles tendinopathy was based on an insidious onset of Achilles tendon pain, a tender nodule localized to the region 2 to 6 centimeters from the calcaneal insertion, and an ultrasound examination that excluded a frank tendon tear. The diagnosis of chronic extensor tendinopathy at the elbow was based on an insidious onset of lateral elbow pain, tenderness localized to the lateral humeral epicondyle and extensor carpi radialis brevis tendon, pain in the lateral elbow with resisted wrist or third metacarpophalangeal joint extension, and an ultrasound examination that excluded a frank tendon tear. The diagnosis of chronic supraspinatus tendinopathy was based on positive impingement signs (internal or external rotation), pain with supraspinatus muscle testing, and magnetic resonance imaging (MRI) high signal intensity without frank tear in the supraspinatus tendon.

Patients were excluded if they had tendinopathy of less than three months duration, current pregnancy, previous surgery on the affected limb or tendon, dislocation of the ipsilateral limb joints, distal neurological signs, a local corticosteroid injection in the previous three months, the current use of nitrate medications or phosphodiesterase inhibitors such as Viagra™, a family history of arthritis other than osteoarthritis, or extra-articular features of seronegative arthropathies. Generally, every participating patient could be considered to have persistent active tendinopathy.

The patients were randomly allocated into two groups. One group performed tendon rehabilitation and used the active transdermal patch (one quarter of a 5 mg/24 hour Nitro-Dur™ glyceryl trinitrate patch, Schering-Plough, Australia), and the other group performed tendon rehabilitation and used a placebo transdermal patch (one quarter of a Nitro-Dur™ demonstration patch). The active and placebo patches were indistinguishable from one another. The randomization was controlled by the senior pharmacist at the institution who also supervised the packaging of transdermal patches and their distribution to patients. Both the patients and the clinical examiner were blinded as to which group the patients were in, i.e. double-blind.

The transdermal patches were intact when distributed, and patients were required to cut the patches into quarters prior to application. Patients were also given a supply of paracetamol tablets (500 mg), and were instructed to use them exclusively for any headaches experienced.

Patients were instructed in the application of the patches at their initial visit. They were informed that the dosing regimen was one quarter of a transdermal patch to be applied daily to the skin area closest to the affected tendon. The patches were to be left in situ for 24 hours and then replaced with a new quarter patch. The site of application was demonstrated as over the site of maximal tendon tenderness (region 2 to 6 centimeters from the calcaneal insertion of the Achilles tendon; immediately distal to the lateral humeral epicondyle; and immediately distal to the anteroinferior aspect of the acromion, respectively for each group). Patients were instructed to rotate the patch application site around this point with each new patch application for the six-month study duration in an effort to minimize application site irritation.

At the initial clinical assessment, all patients were instructed in the performance of a tendon specific rehabilitation program. The aim of this program was to encompass the current non-operative management for tendinopathy, and involved the following regimens. Rehabilitation for Achilles tendon was as follows: (a) rest from aggravating activities in the early stages (particularly repetitive weight-bearing activities such as walking, running, and jumping), (b) the use of 1-1.5 centimeter heel raises, (c) prolonged daily static stretching of the gastrocnemius and soleus musculature, and (d) an eccentric calf muscle strengthening program. Rehabilitation for the extensor carpi radialis brevis tendon was as follows; (a) rest from aggravating activities in the early stages (particularly strong gripping and repetitive forearm and wrist movements), (b) the early continuous use of a forearm counterforce brace, (c) prolonged daily static stretching of the wrist extensor musculature, and (d) a muscle strengthening program initially using isometric exercise and progressing to isotonic exercises of both concentric and eccentric types. For the supraspinatus and rotator cuff tendons, rehabilitation was as follows: (a) early rest from aggravating activities (especially heavy lifting, overhead and behind the back activities), (b) daily range of motion exercises and stretching of the posterior shoulder capsule and pectoral muscles, and (c) muscle strengthening with scapular retraction exercises and closed kinetic chain isometric exercises, gradually progressing to dynamic open kinetic chain isotonic resistance exercises.

In addition, at the initial visit and at all subsequent visits, the patient was required to complete a tendon specific symptom assessment sheet using verbal descriptor scales to rate the severity (0-4: none, mild, moderate severe, very severe) of their tendon pain with activity, at rest, and at night. This verbal descriptor questionnaire has been validated as a reliable measure of monitoring pain that is responsive to clinical change, and these three patient-rated pain scores were used as trial outcome measures.

A single examiner assessed all patients and recorded information on clinical outcome measures. All clinical assessments were repeated at week 0, 2, 6, 12, and 24 with an identical format. Records of headaches, paracetamol use, and compliance with patch application and the tendon rehabilitation program were also made at these scheduled visits. Patients were excluded from the trials for non-compliance at any two visits.

For the Achilles tendinopathy trial the outcome measures were as follows: (a) the degree of Achilles tendon tenderness, as assessed using a four point scale (0-3: none, mild, moderate, severe tenderness), (b) patient-rated analogue pain score after the single leg stationary 10 hop test (rated 0-10), (c) measurement of ankle plantarflexor mean peak force (in Newtons) using a resisted footplate device, and (d) measurement of total ankle plantarflexor work using the ORI-ASTS (in Newtons per 20 seconds). This valid and reliable resisted footplate test involved seating the patient with the foot secured to the footplate, and required them to perform a 20 second effort of repeated ankle plantarflexion and dorsiflexion. The footplate was linked to a load cell and the readings were stored directly on computer hard drive using LabView 5.1 biomechanical software (National Instruments, California, U.S.A.).

For the extensor tendinopathy trial the clinical outcome measures were as follows: (a) assessment the level of local epicondylar and proximal common extensor tendon tenderness using a 4 point scale (0-3: none, mild, moderate, severe tenderness), (b) hand-held dynamometer measurement of resisted 3rd finger metacarpophalangeal extension with a fully extended elbow (in Newtons), (c) measurement of wrist extensor tendon mean peak force (in Newtons) using a modified chair pick-up test, and (d) measurement of total work using the ORI-TETS (in Newtons per 10 seconds). This modified chair pick up test has demonstrated reliability and validity for testing extensor tendinopathy patients, and was performed with the elbow flexed to ninety degrees, and a vertically oriented hand board gripped palm downwards and pulled superiorly for a maximal 10 second effort. The hand board was linked in series with a load cell and the readings stored directly on computer hard drive using LabView 5.1 biomechanical software (National Instruments, California, U.S.A.).

For the supraspinatus tendinopathy trial the clinical outcome measures were as follows: (a) assessment of anteroinferior subacromial tenderness (0-3: no tenderness, mild, moderate, severe), (b) visually assessed passive shoulder range of motion in abduction, forward flexion, external rotation (in degrees), and internal rotation (hand behind back; in centimetres from vertebra prominens), (c) hand-held dynamometer measurement of muscle force in “empty can” position (90 degrees abduction in scapular plane with full internal rotation), adduction, external rotation, internal rotation, and subscapularis push-off (in Newtons), and (d) impingement tests in internal rotation (Hawkins test) and external rotation (0-1: negative or positive).

Outcome measures were analyzed with Sigmastat 2.0 statistical software (Jandel Scientific, California, U.S.A) using Mann-Whitney rank sum tests to compare differences between groups, and using the Wilcoxon sign rank test to compare differences within the groups. The level of significance was defined at p=0.05. A Chi square analysis of patient reported symptom outcomes at week 24 was performed. Effect size estimates were calculated by dividing the mean z-score, calculated from all outcome measures at week 24, by the square root of the sample size to given a general measure of the overall effect of the patch on pain, tendon force and function.

Analysis of the clinical trial outcome measures for all three trials determined that the data was not normally distributed. Mann-Whitney rank sum analysis compared the glyceryl trinitrate groups with the placebo groups for the individual specific tendinopathies. The significant results are summarized in Table I.

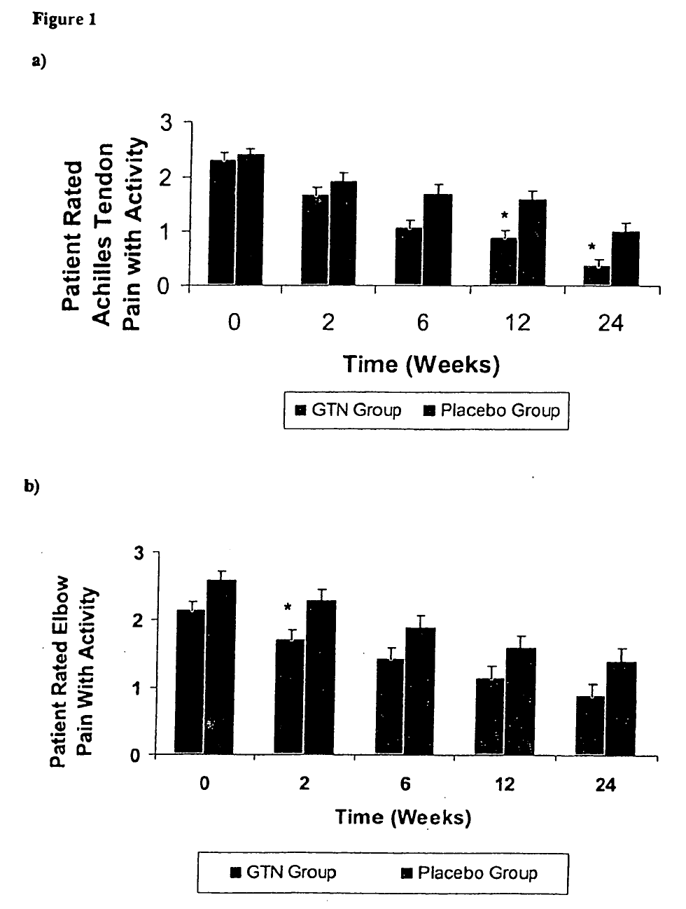

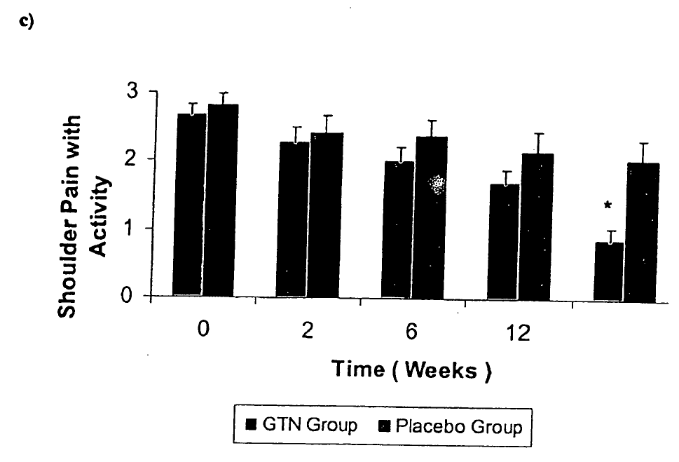

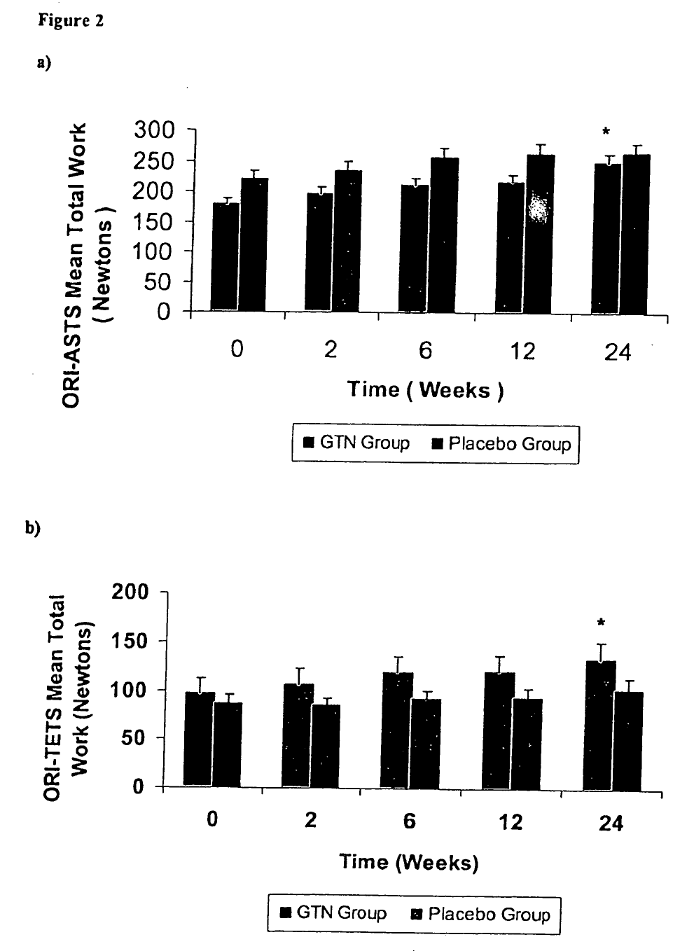

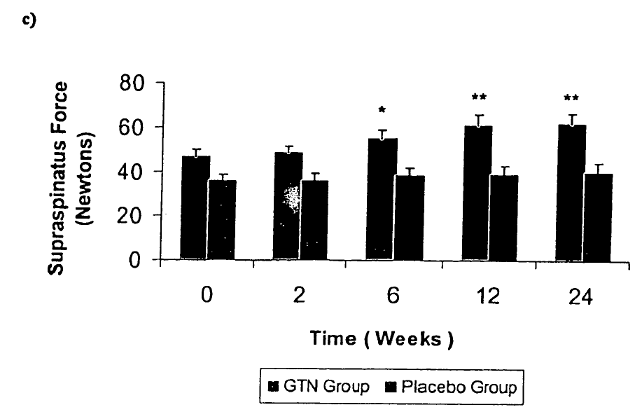

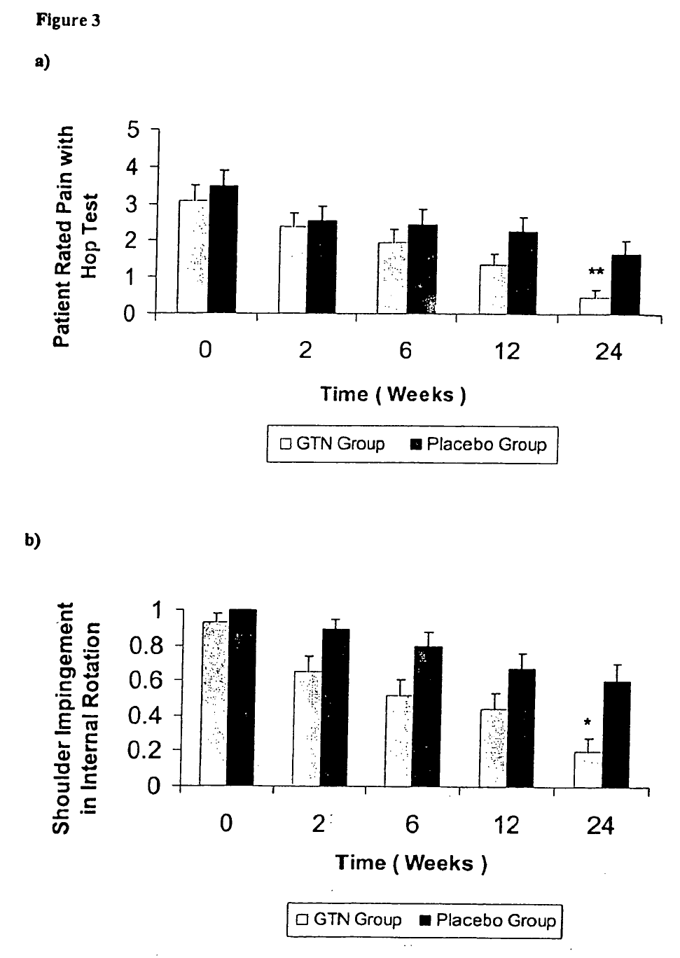

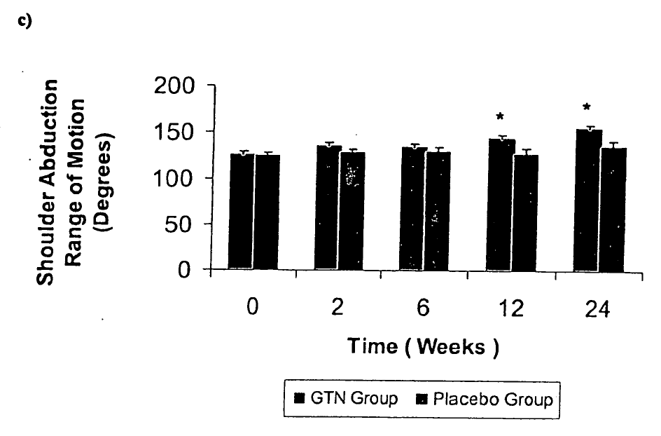

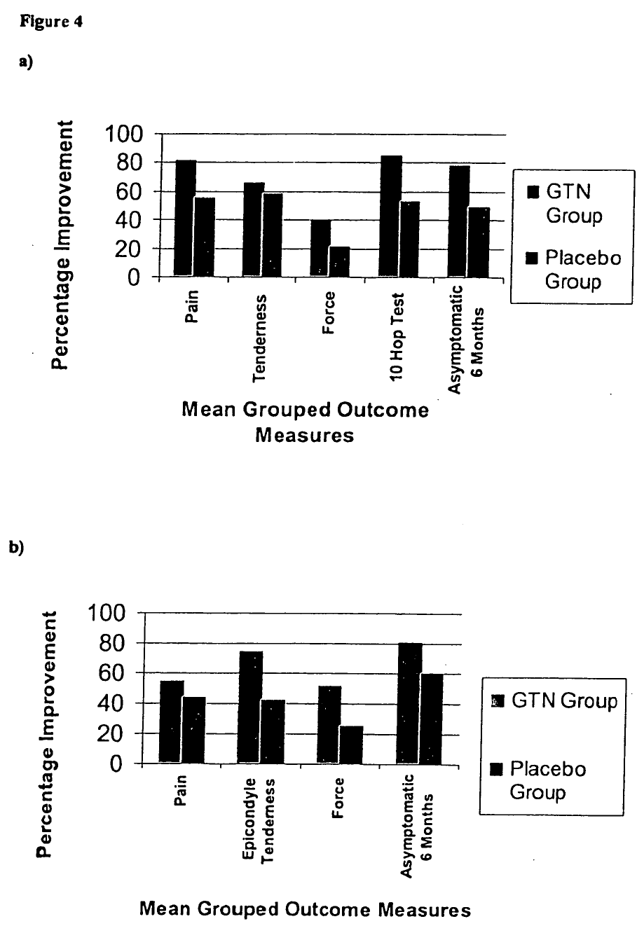

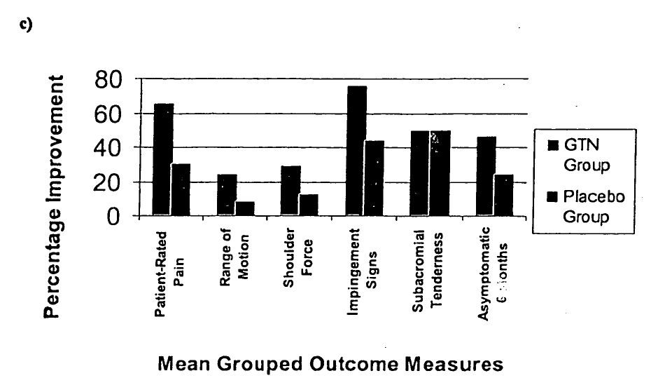

Pain outcome measures in the non-insertional Achilles tendinopathy trial demonstrated that the glyceryl trinitrate group compared to the placebo group had a significant decrease in Achilles tendon pain with activity at week 12 (p=0.02) and at week 24 (p=0.03) (FIG. 1a), and a significant decrease in night pain at week 12 (p=0.04). Pain outcome measures in the extensor tendinopathy trial the glyceryl trinitrate group also showed a significant decrease in elbow pain with activity at week 2 when compared to the placebo group (p=0.01) (FIG. 1b). Pain outcome measures in the supraspinatus tendinopathy trial similarly showed that the glyceryl trinitrate group compared to the placebo group had a significant decrease in shoulder pain with activity at week 24 (p=0.01) (FIG. 1c), a significant decrease in night pain at week 12 (p=0.03) and at week 24 (p=0.01), and a significant decrease in rest pain at week 12 (p=0.04) and week 24 (p=0.03).

Mann-Whitney rank sum tests comparing tendon tenderness between groups in the clinical trials showed significantly less Achilles tenderness at week 12 (p=0.02), and significantly less lateral epicondylar tenderness at week 6 (p=0.02) and at week 12 (p=0.02), in the glyceryl trinitrate group.

TABLE I

| Trial | | Achiles | Elbow | Shoulder |
|---|---|---|---|---|
| Parameters | | (N = 65) | (N = 86) | (N = 53) |
| Improved Patient | | 29% | 21% | 22% |
| Outcomes | | | | |
| Effect Size | | 0.14 | 0.12 | 0.26 |
| Pain | Activity | Decreased | Decreased | Decreased |
| Out- | | Week 12/24 | Week 2 | Week 24 |
| comes | Night | Decreased | — | Decreased |
| | | Week 12 | | Week 12/24 |
| | Rest | — | — | Decreased |
| | | | | Week 12/24 |

| | | | |
|---|---|---|---|
| Force Outcomes | Increased Mean | Increased Mean | Increased |
| | Total Work Week | peak force and | supraspinatus, |
| | 24 | mean total | ER, IR, adduction |
| | | work Week 24 | subscapularis |
| | | | Week 12/24 |
| Tenderness | Decreased Week | Decreased | — |
| Outcomes | 12 | Week 6/12 | |
| Functional | Increased Hop | — | Increased |
| Outcomes | Test Week 24 | | abduction, IR |
| Measures | | | ROM Week 24, |
| | | | Decreased IR |
| | | | Impingement |
| | | | Week 24 |

Table I summarizes results of the topical glyceryl trinitrate clinical trials on Achilles tendinopathy, extensor tendinopathy at the elbow, and supraspinatus tendinopathy, including patient outcomes, effect sizes, and demonstrated significant differences in trial outcome measures.

Regarding force outcome measures, the glyceryl trinitrate group compared to the placebo group in the non-insertional Achilles tendinopathy trial had a significant increase in ORI-ASTS measured mean plantarflexion total work from baseline levels at week 24 (p=0.04) (FIG. 2a), and in the extensor tendinopathy trial had a significant increase in ORI-TETS measured mean peak force at week 24 (p=0.03) and a significant increase in ORI-TETS mean total work at week 24 (p=0.03) (FIG. 2b). In the supraspinatus tendinopathy trial, the glyceryl trinitrate group had significantly increased supraspinatus force at week 6 (p=0:01), week 12 (p=0.001) and week 24 (p=0.001) (see FIG. 2c), significantly increased external rotation force at week 12 (p=0.01) and week 24 (p=0.004), significantly increased internal rotation force at week 12 (p=0.01) and week 24 (p=0.01), significantly increased subscapularis force at week 2 (p=0.01), week 12 (p=0.02) and week 24 (p=0.01), and significantly increased adduction force at week 12 (p=0.01) and week 24 (p=0.04).

The glyceryl trinitrate group compared to the placebo group in the non-insertional Achilles tendinopathy trial also had a significant decrease in pain scores after the 10 hop test at week 24 (p=0.005) (FIG. 3a) in regard to functional outcome measures, and in the supraspinatus tendinopathy trial had a significant decrease in impingement in internal rotation at week 24 (p=0.02) (FIG. 3b), a significant increase in passive shoulder abduction range of motion at week 12 (p=0.03) and week 24 (p=0.02) (FIG. 3c), and a significant increase in shoulder internal rotation range of motion at week 24 (p=0.04).

In the Achilles tendinopathy trial patient reported outcomes at week 24 showed that 78% of patients in the glyceryl trinitrate group had excellent improvement (asymptomatic with activities of daily living) over the course of the trial compared with patient ratings of 49% excellent in the placebo group (FIG. 4a). In the extensor tendinopathy trial patient reported outcomes at week 24 showed that 81% of patients in the glyceryl trinitrate group had excellent improvement over the course of the trial compared with patient ratings of 60% excellent in the placebo group (FIG. 4b). In the supraspinatus tendinopathy trial patient reported outcomes at week 24 showed that 46% of patients in the glyceryl trinitrate group had excellent improvement over the course of the trial compared with patient ratings of 24% excellent in the placebo group (FIG. 4c). Chi square analyses comparing outcomes between the two groups revealed that the glyceryl trinitrate group had a significantly increased (p=0.001) chance of being asymptomatic with activities of daily living at 24 weeks in all three clinical trials (Achilles tendinopathy trial: p=0.001, number needed to treat (NNT)=3.4), (extensor tendinopathy trial: p=0.005, NNT=4.8), (supraspinatus tendinopathy trial: p=0.007, NNT=4.5).

Effect size estimations at week 24 in the three clinical trials were for glyceryl trinitrate in the treatment of Achilles tendinopathy 0.14 (95% Cl 0.09-0.19), for glyceryl trinitrate in the treatment of extensor tendinopathy at the elbow 0.12 (95% Cl 0.06-0.19), and for glyceryl trinitrate in the treatment of supraspinatus tendinopathy 0.26 (95% Cl 0.19-0.32).

In the clinical trials the majority of patients in the glyceryl trinitrate group experienced headache as a side-effect (Table II), however, only in the supraspinatus tendinopathy trial was there a significant increase in the number of days affected by headache (p=0.001). There were significant increases in the total amount of paracetamol required for headache treatment in the glyceryl trinitrate group for the Achilles tendinopathy trial (p=0.001), and the supraspinatus tendinopathy trial (p=0.001).

Within the three clinical trials there were no significant differences between groups in drop-out rates or trial completion rates (Table II). The patients that were discontinued from the clinical trials, mainly for side-effects of headache or application site rash, were all receiving topical glyceryl trinitrate.

TABLE II

| | | Achilles | | Shoulder |
|---|---|---|---|---|
| Trial Parameters | | (N = 65) | Elbow (N = 86) | (N = 53) |
| Trial Completion Rate | | GTN 84% | GTN 81% | GTN 88% |
| | | Placebo 94% | Placebo 91% | Placebo 93% |
| Discontinuations (All | | Rash, | Rash 2, | Headache 2 |
| GTn Group) | | Headache 1 | Headache 2 | |
| Drop-outs | | GTN 2 | GTN 3 | GTN 1 |
| | | Placebo 1 | Placebo 4 | Placebo 2 |
| Headache | Total | GTN 85 (53%) | GTN 136 (63%) | GTN 127 |
| (Days) | | | | (76%) |
| | | Placebo 101 | Placebo 166 | Placebo 37 |
| | | (45%) | (58%) | (33%) |
| | Average | GTN 5 | GTN 5 | GTN 6 |
| | | Placebo 7 | Placebo 7 | Placebo 74 |
| | Median | GTN 4 | GTN 3 | GTN 4 |
| | | Placebo 3 | Placebo 1 | Placebo 0 |
| Paracetamol | Total | GTN 237 | GTN 214 | GTN 138 |
| | | Placebo 46 | Placebo 250 | Placebo 69 |
| | Average | GTN 14 | GTN 8 | GTN 7 |
| | | Placebo 3 | Placebo 10 | Placebo 8 |
| | Median | GTN 10 | GTN 4 | GTN 2 |
| | | Placebo 0 | Placebo 0 | Placebo 0 |
| Other Noted | GTN | Rash 16%, | Rash 21%, | Rash 8% |
| Side-Effects | | Increase | Increase Axillary | |
| | | Tinnitus (3%) | Sweating 2% | |
| | Placebo | Rash 12% | Rash 9% | Rash 7% |

| | | | |
|---|---|---|---|
| No Side Effects | GTN 44% | GTN 35% | GTN 30% |
| | Placebo 45% | Placebo 33% | Placebo 59% |

Table II summarizes results of the topical glyceryl trinitrate clinical trials on Achilles tendinopathy, extensor tendinopathy at the elbow, and supraspinatus tendinopathy, including trial completion rates, discontinuations, drop-outs, and noted side-effects.

These three randomized, double blind, placebo controlled clinical trials demonstrate that continuous 1.25 mg/24 hour topical glyceryl trinitrate application used as therapy for chronic tendinopathies can result in significantly decreased tendon pain with activity, significantly decreased tendon tenderness, significantly improved functional measures, and significantly improved patient outcomes when compared with tendon rehabilitation alone.

At the completion of the clinical trials 21-29% more patients in the glyceryl trinitrate-treated group than the placebo group were asymptomatic with activities of daily living, and rated their specific tendon as excellent. From these results the number of patients needed to treat (NNT) to obtain a positive outcome can be calculated. For every 3.4 chronic Achilles tendinopathy patients, every 4.8 extensor tendinopathy patients, and every 4.5 supraspinatus tendinopathy patients treated with topical glyceryl trinitrate therapy, one patient will have an excellent result at 24 weeks that would not have occurred with placebo treatment.

The mean estimated effect sizes at week 24 for the three clinical trials ranged from 0.12-0.26, which are equivalent to binomial effect size displays, or changes in patient success rates of 12-26%. This effect size range is comparable to the 21-29% improvement in patient rated outcomes noted with topical glyceryl trinitrate therapy. These closely related parallel outcomes calculated from very different sources (patient rated outcomes versus all trial outcome measures) apparently quantify the estimated size of the effect of topical glyceryl trinitrate in treating chronic tendinopathies. While the overall outcomes from the three clinical trials appear closely related, the individual outcome measures require a closer analysis to determine the effects of topical glyceryl trinitrate on tendons.

Within the clinical trials the outcome measure of tendon pain with activity was significantly improved in the glyceryl trinitrate groups in all three trials, although the timing of the improvement varied from early in extensor tendinopathy, to late with non-insertional Achilles tendinopathy and supraspinatus tendinopathy. The reason for this may be due to the immediately subcutaneous position of the lateral humeral epicondyle and extensor carpi radialis brevis tendon. Despite the fact that the Achilles tendon is also subcutaneous, it is less regular in contour (especially with any variation in patch application to either the medial or lateral aspect of the tendon).

An analysis of the between group means at week 0 compared with week 24 demonstrated that the glyceryl trinitrate group patient-rated pain scores (with activity, at night, and at rest) for the trials decreased by an average of 65% (range 64-67%), while the placebo group scores for the trials decreased by an average of 30% (range 27-33%) (FIGS. 4a-c). These results suggest that topical glyceryl trinitrate may have a pain modulation effect in chronic tendinopathies, although the effect appears to differ in timing between specific tendon sites. Possible mechanisms for this effect include increased blood supply to the region due to local vasodilatation, increased clearance of local inflammatory mediators or bioactive proteins such as substance P, or local effects on neural structures, neovascularisation, or apoptosis that may lead to modulation of tendon pain.

Across all three clinical trials there were significant increases in force outcome measures in the glyceryl trinitrate groups at the week 24 stage, with the Orthopaedic Research Institute-Ankle Strength Testing System (ORI-ASTS) and Tennis Elbow Testing System (ORI-TETS), demonstrating increased mean total work, and all dynamometer resisted force measurements for the rotator cuff tendons demonstrating significant increases. These outcome measures have demonstrated excellent intra-rater reliability and validity in testing patients with specific chronic tendinopathies. An analysis of the between group means at week 0 compared with week 24 demonstrated that the glyceryl trinitrate group force outcome measures for the trials increased by an average of 37% (range 33-38%), while the placebo group scores for the trials increased by an average of 16% (range 11-20%). These results suggest that topical glyceryl trinitrate may have an effect on tendon that increases force measures in chronic tendinopathies. This may be a direct effect on tendon metabolism or fibroblasts possibly increasing collagen synthesis and remodeling or an indirect effect due to possible pain modulation.

In the glyceryl trinitrate groups functional outcome measures were significantly increased at week 24 relative to the placebo group in all three clinical trials. These functional tests included the 10 hop test for non-insertional Achilles tendinopathy, the ORI-TETS mean peak force and mean total work for extensor tendinopathy, and shoulder passive range of motion in abduction and in internal rotation, as well as shoulder impingement in internal rotation for supraspinatus tendinopathy. All of these measures reflect important functional characteristics of the tendons involved: hopping involves Achilles tendon loading through push-off and landing as used in running and jumping; wrist extensor tendon peak force and total work measured with a modified chair pick-up test (ORI-TETS) as seen when lifting heavy objects; shoulder range of motion in abduction when utilizing supraspinatus function for overhead activities, shoulder range of motion in internal rotation as used with toileting and dressing, and shoulder impingement in internal rotation which is a common cause of shoulder pain in patients with supraspinatus tendinopathy and may perpetuate the “vicious cycle” of rotator cuff tendon injury and dysfunction. These results indicate that glyceryl trinitrate may modulate tendon function, and again this may be through direct or indirect effects on tendon, but correlates with the results of both decreased pain and increased force suggesting increased control of movement.

Clinical assessment of tendon tenderness revealed significant decreases in the glyceryl trinitrate groups at week 12 in both the Achilles and elbow tendinopathy clinical trials. There were no significant differences in the supraspinatus tendinopathy trial. These results may be due to the subcutaneous nature of the Achilles and extensor carpi radialis brevis tendons relative to the deeper supraspinatus tendon. The decreased tenderness precedes any significant improvements in force and function measurements (and may represent a manifestation of pain modulation prior to any structural alteration in tendon allowing increased force production.)

The number of patients discontinued during the course of the clinical trials ranged from 4-6% of clinical trial patients, these patients were all in the glyceryl trinitrate groups, and they were discontinued for recognized side-effects of headache or application site rash. One patient was discontinued for recurrent facial flushing, which was reversible on discontinuation of the medication. This patient was a type 2 diabetic and it was felt that this side-effect was caused by arteriolar dilatation (Table II).

The trial completion rate for the glyceryl trinitrate group ranged from 81-88% and the placebo group ranged from 91-94%. There was no significant difference between groups in regard to completion, or drop-out, rates between groups. If discontinued patients were excluded from this analysis, the trial completion rates differed by less than 4%. The high completion rate amongst groups may be due to the thorough explanation of requirements for the clinical trial prior to entry, frequent assessment visits, relatively low side-effect profile of the medication, or the personalities of patients entering clinical trials.

Headache was the most frequent side-effect and in the glyceryl trinitrate group and ranged from 53-76% of patients, with an average number of days of headache ranging from 5-6 days, and the median number of days of headache ranging from 3-4 days. 72% of headaches in the glyceryl trinitrate groups occurred within the first two weeks of the trial. The percentage of patients experiencing headache in these clinical trials was higher than that reported in the literature of 18-68% for dosages of 5 mg/24 hour. It is difficult to understand the reasons for this, especially as the dosing regime used in the clinical trials was a continuous low dose of 1.25 mg-2.5 mg/24 hours, but this may be due to better patient reporting of side-effects, since patients were required to complete a headache diary which was checked for compliance. The placebo groups also reported high rates of headache ranging from 33-58% of patients, with an average number of days of headache ranging from 4-7 days, and the median number of days of headache ranging from 0-3 days.

Patients in the clinical trials were supplied with paracetamol (Tylenol™) tablets for exclusive use with potential headaches. In the glyceryl trinitrate groups the total paracetamol usage ranged from 138-237 tablets, with an average of 7-14 tablets, and a median of 2-10 tablets. In the placebo groups the total paracetamol usage ranged from 69-250 tablets, with an average of 3-10 tablets, and a median of 0 tablets. There were significant between-group differences in the reported rate of headache and the average number of headaches experienced in the supraspinatus tendinopathy trial, but not in the other clinical trials. There was also significant between-group differences in the total amount of paracetamol used in the Achilles tendinopathy and supraspinatus tendinopathy trials. The higher rates of headache in the supraspinatus tendinopathy trial may be due to the glyceryl trinitrate patch application site being closer to both the cardiac and cerebral circulation than either the extensor tendinopathy or Achilles tendinopathy trials, possibly leading to greater systemic and local vasodilation. Despite the high rates of headache in the supraspinatus tendinopathy trial, the use of paracetamol was lower than in either of the other clinical trials. It should be noted that, in general, the glyceryl trinitrate group experienced more severe headaches than the placebo group, as evidenced by 1-2 patients in each clinical trial discontinued due to this side-effect and the placebo group median use of paracetamol being zero.

Another common side-effect of topical glyceryl trinitrate was application site rash and in the glyceryl trinitrate groups the number of patients experiencing rash ranged from 8-21%. This compared with rates in the placebo groups ranging from 7-12%. Reports in the literature for glyceryl trinitrate dosages of 5 mg/24 hour note rash occurred in 16-38% of patients, and these side-effect rates are comparable with those reported in these clinical trials. There was a greater severity of rash in the glyceryl trinitrate groups compared to the placebo groups as evidenced by a total of five patients discontinued due to this side-effect.

Other side-effects that were reported included: an increase in pre-existing tinnitus, increased ipsilateral axillary sweating, and a perception of apprehension. None of these were severe, and all were reversible on discontinuation of the medication at the conclusion of the clinical trials. The number of patients in the glyceryl trinitrate groups that experienced no side-effects ranged from 30-44%, while those in the placebo groups ranged from 33-59%.

These clinical trials investigating topical glyceryl trinitrate donation with tendon rehabilitation demonstrated improved patient rated pain scores, increased tendon force measures, improved functional measures, and improved patient outcomes relative to tendon rehabilitation alone in the treatment of chronic overuse tendinopathies. Glyceryl trinitrate have a long history of therapeutic use in humans, has a known side-effect profile with no irreversible effects, and now has clinically demonstrated efficacy in modulating pain, force measures, functional measures, and patient outcomes at six months in specific chronic overuse tendinopathies. These studies it establish that transdermal glyceryl trinitrate is effective in treating specific overuse tendinopathies in mammals and especially in humans.

### Example 2

A three year follow-up study of the experiment presented in Example 1 was performed. The study measured and compared the outcome measures of those patients who received treatment with a 1.25 mg glyceryl trinitrate patch in the original study (Example 1) to those who received a placebo. Among the 52 patients in follow-up study (24 on the active patch and 32 on placebo), 15 had bilateral involvement (7 on the active patch and 8 on placebo). Results from the follow-up study are presented below. The results are presented in two sets. The follow-up study used the same outcome measures as were used in the original parent study.

The first set compares the results of the 68 heels and the second set results are shown for the 52 patients by averaging results for patients with bilateral involvement. Analyses were performed as analyses of covariance, with the baseline assessment as the covariate, to adjust the results for baseline differences. Mean results shown in Tables III-XIII below are adjusted for the baseline values.

TABLE III
Pain With Activity—All Heels

| | | Active | Placebo | |
|---|---|---|---|---|
| Assessment | | (N = 32) | (N = 36) | P-Value |
| 24 | weeks | 0.52 | 0.78 | 0.22 |
| 3 | years | 0.16 | 0.50 | 0.059 |

TABLE IV
Pain With Activity—Average Result Used for Bilateral Patients

| | | Active | Placebo | |
|---|---|---|---|---|
| Assessment | | (N = 24) | (N = 28) | P-Value |
| 24 | weeks | 0.44 | 0.86 | 0.09 |
| 3 | years | 0.15 | 0.51 | 0.09 |

TABLE V
Hop Test—All Heels

| | | Active | Placebo | |
|---|---|---|---|---|
| Assessment | | (N = 32) | (N = 36) | P-Value |
| 24 | weeks | 0.61 | 1.57 | 0.026 |
| 3 | years | 0.36 | 0.96 | 0.07 |

TABLE VI
Hop Test—Average Result Used for Bilateral Patients

| | | Active | Placebo | |
|---|---|---|---|---|
| Assessment | | (N = 24) | (N = 28) | P-Value |
| 24 | weeks | 0.44 | 0.86 | 0.09 |
| 3 | years | 0.15 | 0.51 | 0.09 |

TABLE VII
Night Pain—All Heels

| | | Active | Placebo | |
|---|---|---|---|---|
| Assessment | | (N = 32) | (N = 36) | P-Value |
| 24 | weeks | 0.19 | 0.30 | 0.36 |
| 3 | years | 0.10 | 0.27 | 0.07 |

TABLE VIII
Night Pain—Average Result Used for Bilateral Patients

| | | Active | Placebo | |
|---|---|---|---|---|
| Assessment | | (N = 24) | (N = 28) | P-Value |
| 24 | weeks | 0.15 | 0.34 | 0.11 |
| 3 | years | 0.07 | 0.29 | 0.06 |

TABLE IX
Pain at Rest—All Heels

| | | Active | Placebo | |
|---|---|---|---|---|
| Assessment | | (N = 32) | (N = 36) | P-Value |
| 24 | weeks | 0.29 | 0.41 | 0.35 |
| 3 | years | 0.10 | 0.35 | 0.07 |

TABLE X
Pain at Rest—Average Result Used for Bilateral Patients

| | | Active | Placebo | |
|---|---|---|---|---|
| Assessment | | (N = 24) | (N = 28) | P-Value |
| 24 | weeks | 0.26 | 0.46 | 0.20 |
| 3 | years | 0.10 | 0.39 | 0.08 |

TABLE XI
Tenderness—All Heels

| | | Active | Placebo | |
|---|---|---|---|---|
| Assessment | | (N = 32) | (N = 36) | P-Value |
| 24 | weeks | 0.69 | 0.83 | 0.49 |
| 3 | years | 0.35 | 0.66 | 0.031 |

TABLE XII
Tenderness—Average Result Used for Bilateral Patients

| | | Active | Placebo | |
|---|---|---|---|---|
| Assessment | | (N = 24) | (N = 28) | P-Value |
| 24 | weeks | 0.70 | 0.90 | 0.43 |
| 3 | years | 0.33 | 0.66 | 0.052 |

TABLE XIII
RTS

| | Active | Placebo | |
|---|---|---|---|
| | (N = 24) | (N = 28) | |
| Assessment | n(%) | N(%) | P-Value |
| 3 years | 21(87.5%) | 20 (71.4%) | 0.16 |

Patients on the active patch exhibited numerically better results than those on the placebo for each assessment both at 24 weeks and at 3 years. Three-year results achieved statistical significance (p<0.05), or were very close for many of the endpoints assessed, as shown in Tables 3-13.

### Example 3

A 35 year old male patient suffering from persistent active tendinopathy of the left Achilles tendon applies a transdermal patch delivering 30 mcg/hr nitroglycerin for a period of 8 weeks. The patient experiences a reduction of pain and tenderness of the ankle by day 2 of therapy, which progressively improves over the treatment period. Eight weeks following the discontinuation of glyceryl trinitrate administration to the affected tendon, a patient would continue to feel decreased pain and tenderness of the ankle.

### Example 4

A 35 year old female patient suffering de Quervain's tendinopathy in the right extensor tendons of the thumb applies a transdermal patch delivering 10 mcg/hr nitroglycerin for a period of 24 weeks. This condition can be caused due to the arrival of a new baby and the consequent carrying as an unusual daily activity, and as is common, physical therapy and intermittent use of a wrist splint may provide little relief of symptoms. A decrease in pain within one day of beginning application of a nitroglycerin-containing transdermal patch is typical, and the reduced pain continues throughout the administration period. 2 years after the administration of the nitroglycerin is stopped, the patient would continue to have reduced pain, which can be demonstrated by a negative Finklestein test.

### Example 5

A 65 year old male patient suffering from chronic tennis elbow applies a transdermal patch delivering 60 mcg/hr nitroglycerin for a period of 2 weeks. Upon application, the patient experiences a moderate decrease in pain upon elicitation at the end of the treatment period as assessed by grip strength and resisted wrist dorsiflexion. The reduction of symptoms continues during the administration period, and continues for a period of at least two weeks after the discontinuance of the nitroglycerine administration.

What is claimed is:

1. A method of treating a subject experiencing persistent active tendinopathy, comprising administering to a skin site proximate an affected tendon of said subject a glyceryl trinitrate-containing transdermal patch for an administration period of from 1 to 52 weeks, said transdermal patch being configured to deliver glyceryl trinitrate at a rate of from 5 mcg/hr to about 85 mcg/hr, wherein clinical and therapeutic effects of the treatment of the persistent active tendinopathy continue for a durability of effect period of at least as long as the administration period.

2. The method of claim 1, wherein the administration period is from about 4 to about 24 weeks.

3. The method of claim 2, wherein the durability of effect period is from about 3 to about 30 months.

4. The method of claim 1, wherein the administration period is from about 6 to about 12 weeks.

5. The method of claim 1, wherein the administration period is about 8 weeks.

6. The method of claim 1, wherein the administration period is at least about 24 weeks.

7. The method of claim 6, wherein the durability of effect period is at least about 18 months.

8. The method of claim 6, wherein the durability of effect period is at least about 30 months.

9. The method of claim 1, wherein the clinical and therapeutic effects are selected from the group consisting of reduced pain upon activity, reduced night pain, reduced pain at rest, reduced tenderness, improved strength, improved function, increased range of motion, and combinations thereof.

10. The method of claim 1, wherein the transdermal patch is configured to deliver glyceryl trinitrate at a rate of from about 15 mcg/hr to about 75 mcg/hr.

11. The method of claim 1, wherein the transdermal patch is configured to deliver glyceryl trinitrate at a rate of from about 30 mcg/hr to about 65 mcg/hr.

12. The method of claim 1, wherein the persistent active tendionopathy is chronic.

13. The method of claim 1, wherein the persistent active tendinopathy is tendinosis.

14. The method of claim 1, wherein the persistent active tendinopathy is overuse tendinopathy.

15. The method of claim 1, wherein the persistent active tendopathy is tendonitis.

16. The method of claim 1, wherein the persistent active tendinopathy is extensor tendinopathy at the elbow.

17. The method of claim 1, wherein the persistent active tendinopathy is Achilles tendinopathy.

18. The method of claim 1, wherein the persistent active tendinopathy is supraspinatus tendinopathy.

19. The method of claim 1, wherein the subject is a human.

20. The method of claim 1, wherein the persistent active tendinopathy is selected from the group consisting of patellar tendinopathy, quadriceps tendinopathy, hip adductor tendinopathy, common flexor tendinopathy of the elbow, and tendinopathy of the thumb.

21. The method of claim 1, wherein the wherein the glyceryl trinitrate is continuously released over a pre-determined period of time of about 24 hours.

22. The method of claim 1, which further comprises treating the subject with a non-operative rehabilitation regimen comprising at least one of rest, tendon unloading, orthotics, braces, daily prolonged static stretching, or a graduated strengthening exercise program comprising eccentric tendon loading, or combinations thereof, during at least a portion of the time that the mammal is administered the glyceryl trinitrate.

23. The method of claim 1, wherein the method of treating the subject includes a method of reducing or relieving pain caused by the persistent active tendinopathy, wherein relief from pain continues for a durability of effect period of at least as long as the administration period.

25. The method of claim 1, wherein the transdermal patch is a fluid reservoir system.

26. The method of claim 1, wherein the transdermal patch is a drug in adhesive matrix system.

27. A transdermal patch for the delivery of a nitroglycerin, comprising:
a backing layer, and
a glyceryl trinitrate-containing composition supported at least in part by the backing layer, said transdermal patch being formulated to deliver nitroglycerin at from about 5 μg/hour to about 85 μg/hour, said transdermal patch being configured to be administered to a subject during an administration period of from 4 to 52 weeks;
wherein clinical and therapeutic effects provided by the transdermal patch continue against persistent active tendinopathy for a durability of effect period of at least as long as the administration period.

28. The transdermal patch of claim 27, wherein the durability of effect period is from about 3 to about 30 months.

29. The transdermal patch of claim 27, wherein the administration period is from about 6 to about 12 weeks.

30. The transdermal patch of claim 27, wherein the administration period is about 8 weeks.

31. The transdermal patch of claim 27, wherein the administration period is at least about 24 weeks.

32. The transdermal patch of claim 31, wherein the durability of effect period is at least about 18 months.

33. The method of claim 31, wherein the durability of effect period is at least about 30 months.

34. The transdermal patch of claim 27, wherein the clinical and therapeutic effects are selected from the group consisting of reduced pain upon activity, reduced night pain, reduced pain at rest, reduced tenderness, improved strength, improved function, increased range of motion, and combinations thereof.

35. The transdermal patch of claim 27, wherein the transdermal patch is configured to deliver glyceryl trinitrate at a rate of from about 15 mcg/hr to about 75 mcg/hr.

36. The transdermal patch of claim 27, wherein the transdermal patch is configured to deliver glyceryl trinitrate at a rate of from about 30 mcg/hr to about 65 mcg/hr.

37. The transdermal patch of claim 27, wherein the persistent active tendinopathy is selected from the group consisting of patellar tendinopathy, quadriceps tendinopathy, hip adductor tendinopathy, common flexor tendinopathy of the elbow, and tendinopathy of the thumb.

38. The transdermal patch of claim 27, wherein the wherein the glyceryl trinitrate is continuously released over a pre-determined period of time of about 24 hours.

39. The transdermal patch of claim 27, wherein the transdermal patch is either a fluid reservoir system or an adhesive matrix system.
